(19) United States
(12) Patent Application Publication
(10) Pub. No.: US 2022/0370369 A1
(43) Pub. Date: Nov. 24, 2022

(54) FORMULATION OF INTRINSICALLY ACID-RESISTANT VEGETARIAN-BASED AND GELATIN-BASED SOFT GEL CAPSULES FOR PHARMACEUTICAL/ NUTRACEUTICAL PRODUCTS

(71) Applicant: GelEntroCeutics Inc., Winnipeg (CA)

(72) Inventors: Farzad Kobarfard, Winnipeg (CA); Marjan Esfahanizadeh, Winnipeg (CA); Arash Lotfi Seisan, Winnipeg (CA); Hooshang Lotfisisan, Winnipeg (CA)

(21) Appl. No.: 17/711,602

(22) Filed: Apr. 1, 2022

(30) Foreign Application Priority Data
May 18, 2021 (CA) 3119012

(51) Int. Cl. A61K 9/50

(52) U.S. Cl. CPC A61K 9/5057; A61K 9/5047; A61K 9/5026; A61K 9/5036; A61K 9/5089

(57) ABSTRACT

There is provided acid-resistant capsules, and in particular acid-resistant, vegetarian-based, and/or gelatin-based soft gel capsules and method of manufacturing the soft gel capsule. The soft gel capsule has 30% wt. to 45% wt. water; 15% wt. to 19% wt. glycerol; and a gel mass composition comprising a gelling agent and an alkaline agent. The method of manufacturing the capsule involves: dissolving a gelling polymer into water to form an enteric polymer solution; mixing an acid insoluble polymer with an alkali agent to form a film forming polymer; and adding the film forming polymer to the enteric polymer solution while mixing and heating at 70° C. until a gel mass forms.

### REFERENCE TO RELATED APPLICATION

This application claims priority to Canadian Applic. No. 3,119,012, filed on May 18, 2021, the contents of which are incorporated herein by reference.

### FIELD

This invention is in the field of acid-resistant capsules, and in particular acid-resistant, vegetarian-based, and/or gelatin-based soft gel capsules and method of manufacturing the capsules.

### BACKGROUND

Soft gelatin capsules are used to encapsulate water-insoluble liquids dissolved in a non-polar solvent for several reasons, such as masking flavors or unpleasant smell, reducing contamination of the product and protecting the active drug against oxidation. Due to its unique functional capabilities and full compliance with the human body, gelatin is the main ingredient in soft gelatin capsules, commonly known as soft gels.

Oral pharmaceutical dosage forms with gastric resistant properties are employed to avoid degradation of the active substances by the gastric juice and also to reduce gastric irritation caused by the medicine. Enteric coated tablets have been around for decades and provided their advantages to the portions for quite a long time.

### SUMMARY

There is provided a soft gel capsule having: 30% wt. to 45% wt. water; 15% wt. to 19% wt. glycerol; and a gel mass composition comprising a gelling agent and an alkaline agent. The gelling agent may be selected from at least one of: a gelatin and a vegetarian gelling agent. The gelatin may be 25% wt. to 35% wt. The vegetarian gelling agent may comprise at least one of: a tapioca, a pullulan, a hydroxypropyl methylcellulose (HPMC), a hydroxypropyl methylcellulose phthalate (HPMCP), a Eudragit L100, and a Eudragit L30D55. The HPMCP may be 15% wt. to 17.5% wt. The HPMC may be 25% wt. to 27% wt. The Eudragit L100 may be 15% wt. to 18% wt. The Eudragit L100 may be 15% wt. The Eudragit L30D55 may be 15% wt. to 46.5% wt. The Eudragit L30D55 may be 15% wt. to 46.5% wt. The alkaline agent may comprise 2.25 g to 3.95 g of NaOH. The alkaline agent may comprise 3.8 ml to 6.65 ml of a 25% solution of NH4OH. The soft gel capsule may comprise 1.8% wt. triethlycitrate.

There is provided a method of manufacturing a soft gel capsule may comprise: dissolving a gelling polymer into water to form an enteric polymer solution; mixing an acid insoluble polymer with an alkali agent to form a film forming polymer; and adding the film forming polymer to the enteric polymer solution while mixing and heating at 70° C. until a gel mass forms.

The method of manufacturing the soft gel capsule further may further comprise: removing bubbles from the gel mass by maintaining the gel mass at 50° C. for 24-hours. The method of manufacturing the soft gel capsule further may comprise: removing bubbles from the gel mass by placing the gel mass under vacuum for 18-hours. The method of manufacturing the soft gel capsule may comprise: titrating the acid insoluble polymer with concentrations of the alkali agent; determining a first equivalence and a second equivalence from a titration curve, whereby the second equivalence corresponds to when the film forming polymer becomes translucent; and selecting the second equivalence to determine an amount of the alkali agent.

### DESCRIPTION OF THE DRAWINGS

While the invention is claimed in the concluding portions hereof, example embodiments are provided in the accompanying detailed description which may be best understood in conjunction with the accompanying diagrams where like parts in each of the several diagrams are labeled with like numbers, and where:

FIG. 1 is a flow chart of the manufacturing process for the soft gel capsules; and

FIG. 2 is a titration curve of Eudragit L30D55.

### DETAILED DESCRIPTION

Acid resistant solid dosage forms usually have an enteric coating that prevents the disintegration and dissolution in the gastric environment. There are many enteric coated tablets in the pharmaceutical and nutraceutical market. These tablets which are called enteric tablets often deliver the active ingredients straight to the duodenum for intestinal absorption and they are suitable for delivering enzymatic and probiotic formulations. Manufacturing of these tablets comprises of two consecutive separate steps: First tableting and then coating which is performed by application of heat and spraying the coating agents on tablets.

Soft gels are water soluble and heat sensitive and therefore they are deformed and damaged during the coating process which has to be done after the freshly produced soft capsules are made through the encapsulation process. This intensive two-step process adds time and money to the cost of each enteric capsules. Additionally, this coating produces an opaque shell which is less desired by patients.

Soft gelatin capsules are not resistant to the gastric juice and therefore acid-sensitive active pharmaceutical ingredients (API) and/or medications with gastric irritation properties cannot be formulated in a form of soft gel. Acid-resistant capsules have special formulations and ingredients that delay the release of the capsule content. The capsules often have enteric coatings that will not dissolve in the stomach juices.

The delayed release properties are the result of utilization of one of the following compounds as the coating agent: Proprietary polymers, Hypromellose, Zein, Sodium alginate, Shellac, Cellulose acetate trimellitate, Polyvinyl acetate phthalate (PVAP), Cellulose acetate succinate, Cellulose acetate phthalate, Hydroxypropyl methyl cellulose phthalate, and Eudragit®.

Traditionally, enteric soft gels have been prepared by coating with enteric polymers using traditional coating technology for tablets, but coating has disadvantages for soft gels such as unsuccessful adhesion for the enteric polymer onto the soft gelatin shell's inherent flexible nature. Since the coating agents are often dissolved in water and their aqueous solutions are sprayed on the soft gels while applying heat, the soft gels may be damaged and/or deformed during the process. Flaking after the drying step is another challenge for coating soft gels.

There are currently three methods for soft gel coating in pharmaceutical industries: 1—Dipping—the capsules are immersed in solution of acid-resistant polymer and then dried. 2—Pan spray—the capsules are coated in a pan coating machine in which the coating agent is sprayed at a certain temperature on the capsules. The capsules are then tumbled in the pan until they are dry. 3—Fluidized bed coating—In a fluidized bed dryer machine, the capsules are stirred and suspended in the air while coating solution and heat are applied. All three methods are challenging since they all use heat in the process.

Therefore, making gelatin mass with the enteric features which reside in the mass may provide the capability of making soft gelatin capsules with intrinsic acid resistant properties which resides in their shells.

As described herein, a gelatin mass containing an acid-insoluble polymer along with the other additives may make clear enteric soft gelatin capsules with intrinsic acid-resistance properties which resides in the capsule shell.

The polymers contain carboxylic acid functional groups and thus they possess pH-dependent solubility; at high pH, the carboxylic acid groups become ionized and make the polymers dissolve in water. At low pH, carboxylic acid groups are not ionized and render them insoluble in water.

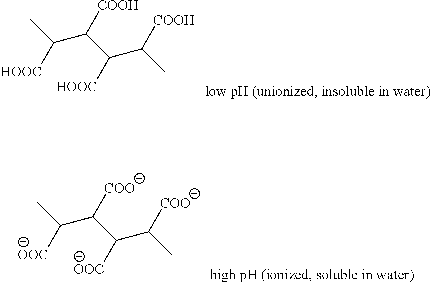

Among the above polymers, Eudragit L, containing an anionic copolymer based on methacrylic acid/ethyl acrylate (1:1) is used in the pharmaceutical industry for coatings.

Chemical structure of Eudragit is shown below.

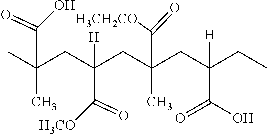

Eudragit L100 is an anionic copolymer based methacrylic acid and methyl methacrylic acid. It has acid value of 315 mg KOH/g of polymer and glass transition temperature greater than 150° C. Targeted drug release area for this polymer is jejunum and dissolves at pH above 6.

Eudragit L30D55 is the aqueous dispersion of anionic polymers with methacrylic acid as a functional group. It is a low viscosity liquid with white color with faint characteristic odor.

Eudragit L30D55 is obtained in the form of aqueous dispersion (30%) whereas Eudragit L100 is an anionic copolymer based on methacrylic acid and ethyl acrylate. Eudragit L100 is a white powder with a faint characteristic odor. Both grades of Eudragit have molecular weight 3,200,000 g/mol, acid value 315 mg KOH/g of polymer. The targeted drug release area for Eudragit L30D55 is duodenum and Eudragit L30D55 dissolves at pH of 5.5. Both Eudragit L100 and Eudragit L30 D55 are used for effective and stable coating with fast dissolution in the upper bowel, controlled release, site specific drug delivery in intestine.

As described herein, combinations of one acid-insoluble polymer and gelatin or vegetarian gelatin (tapioca) or pullulan or hydroxypropyl methylcellulose (HPMC) along with a plasticizer such as glycerin, sorbitol, and/or triethyl citrate have been developed so that a mixture could be used directly for gel making and encapsulation process.

The soft gel capsules which are made using these formulations have an acid-resistant shell which is not disintegrated in acidic condition of stomach and are delivered to the duodenum where the capsules are disintegrated and release their contents.

These soft gelatin capsules have transparent appearance without needing to go through the coating process. The gel which is used in the process of encapsulation is intrinsically acid resistant and therefore the acid resistant soft gel is made in a single step. This one-step process is less time-consuming, less expensive and does not require a second machine for the coating process.

The type of acid-insoluble polymer which is used in combination with gelatin or non-gelatin gel (vegetarian or HPMC) and the proper composition of gelling agent and acid-insoluble polymer and plasticizer may significantly affect a quality of the shell. Encapsulation process is highly sensitive to a bloom of the combined gel and acid-resistant polymer.

Different quantities of acid-resistant polymers may be required for gelatin and non-gelatin to achieve enteric properties.

In order to increase a flexibility and an adhesion of the enteric capsules, an amount of plasticizer was adjusted, based on an acid-insoluble polymer and a gelling agent. Talc was not used as an anti-sticking agent so as to avoid any discoloration of a capsule surface.

Due to the lower glass transition temperature of the polymer in Eudragit L30D, a glidant may be needed to reduce the thickness of the gel.

As described herein, a gel mass composition may be produced without requiring a coating process. The gel mass composition may comprise a gelatin or a vegetarian gelling agent (tapioca and pullulan) or hydroxypropyl methylcellulose phthalate (HPMCP) for manufacturing acid-resistant (enteric) soft gel capsules.

The following are a few examples of percent compositions to achieve enteric properties of soft gel capsules.

### Example 1

| | % wt. |
|---|---|
| Gelatin | 35% |
| Water | 35% |
| Glycerol | 15% |
| HPMCP | 15% |
| NaOH | 3.375 g |

### Example 2

| | % wt. |
|---|---|
| Gelatin | 32.5% |
| Water | 32.5% |
| Glycerol | 17.5% |
| HPMCP | 17.5% |
| NaOH | 3.95 g |

### Example 3

| | % wt. |
|---|---|
| Gelatin | 35% |
| Water | 35% |
| Glycerol | 15% |
| HPMCP | 15% |
| NH₄OH sol. 25% | 5.7 ml |

### Example 4

| | % wt. |
|---|---|
| Gelatin | 32.5% |
| Water | 32.5% |
| Glycerol | 17.5% |
| HPMCP | 17.5% |
| NH₄OH sol. 25% | 6.65 ml |

### Example 5

| | % wt. |
|---|---|
| HPMC | 25% |
| Water | 45% |
| Glycerol | 15% |
| Eudragit L100 | 15% |
| NaOH | 3.375 g |

### Example 6

| | % wt. |
|---|---|
| HPMC | 25% |
| Water | 45% |
| Glycerol | 15% |
| Eudragit L100 | 15% |
| NH₄OH sol. 25% | 5.7 ml |

### Example 7

| | % wt. |
|---|---|
| HPMC | 25% |
| Water | 30% |
| Glycerol | 15% |
| Eudragit L30D55 | 30% |
| NaOH | 2.25 g |

### Example 8

| | % wt. |
|---|---|
| HPMC | 25% |
| Water | 35% |
| Glycerol | 15% |
| Eudragit L30D55 | 25% |
| NH₄OH sol. 25% | 3.8 ml |

### Example 9

| | % wt. |
|---|---|
| HPMC | 27% |
| Water | 31.3% |
| Glycerol | 17.1% |
| Eudragit L30D55 | 19.8% |
| NH₄OH sol. 25% | 4.8% |

### Example 10

| | % wt. |
|---|---|
| Gelatin | 27% |
| Water | 30.4% |
| Glycerol | 18% |
| Triethylcitrate | 1.8% |
| Eudragit L100 | 18% |
| NH₄OH sol. 25% | 4.8% |

### Example 11

| | % wt. |
|---|---|
| Gelatin | 25% |
| Water | 3.9% |
| Glycerol | 19% |
| Triethylcitrate | 1.8% |
| Eudragit L30D5 | 45.5% |
| NH₄OH sol. 25% | 4.8% |

### Example 12

| | % wt. |
|---|---|
| Gelatin | 33.5% |
| Water | 10.2% |
| Glycerol | 17.8% |
| Eudragit L30D5 | 36% |
| NaOH | 2.5% |

### Gel Making Process

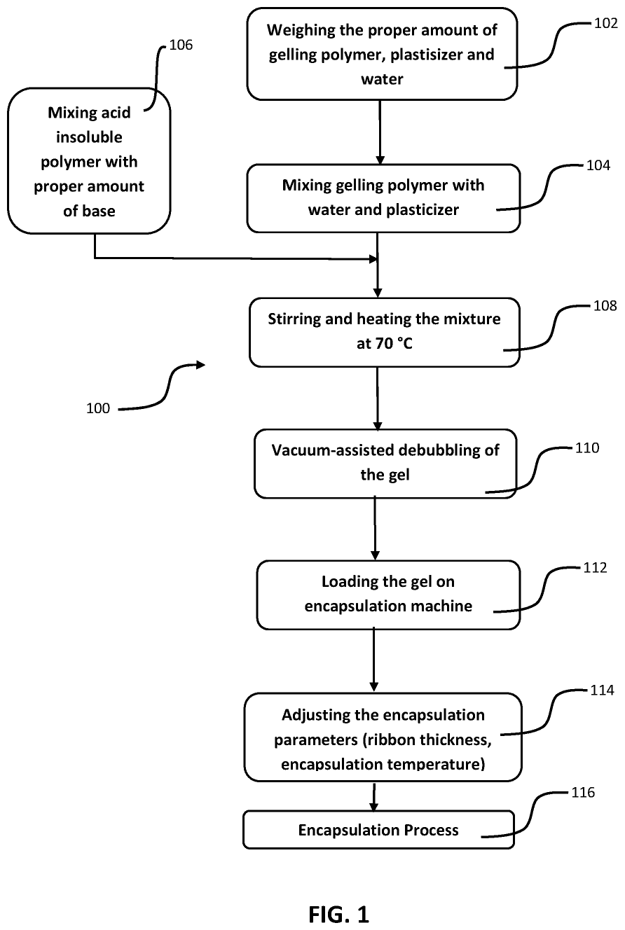

Turning to FIG. 1, the gel making process 100 starts by weighting a specific amount of an acid insoluble polymer, plasticizer, and water at step 102. An acid insoluble polymer may be dissolved in water at step 104 and an alkali agent may then be added to the mixture while stirring at step 106. A film forming polymer (gelatin, HPMC, or vegetarian gelling agent) may be mixed with a plasticizer and water following an addition of the enteric polymer solution. Mixing may be continued for two hours at 70° C. at step 108 and a gel mass may be kept at 60° C. overnight.

The temperature was then brought to 50° C. and the gel mass may be kept at this temperature for 24 hours to remove any bubbles. In an alternative way, the gel mass may be kept under vacuum for 18 hours to remove any bubbles at step 110. The gel mass so obtained may be directly used in encapsulation process. The gel may be loaded on an encapsulation machine at step 112. One or more encapsulation parameters may be adjusted at step 114, such as ribbon thickness, encapsulation temperature, etc.). Then the encapsulation process may be performed at step 116.

An optimum formulation for the manufacturing process for an amount of the alkaline reagent (e.g., NaOH or NH₄OH solution) which may be added to the water insoluble polymer, such as Eudragit L30D55 (methacrylic acid-Ethyl acrylate copolymer). In order to calculate the required amount of alkaline agent, the Eudragit may be titrated as described herein.

The pH titration may be performed using five concentrations (e.g., 0.01, 0.05, 0.1, 0.5, and 1 wt. %) of the Eudragit L30D55 dispersed in water. A volume of 10 ml of the polymer suspension may then be titrated using NaOH solutions of different concentrations according to an equivalent point from 1M to 0.01 M. The titration may be made under stirring, at room temperature, and a pH was measured as a function of the NaOH added volume.

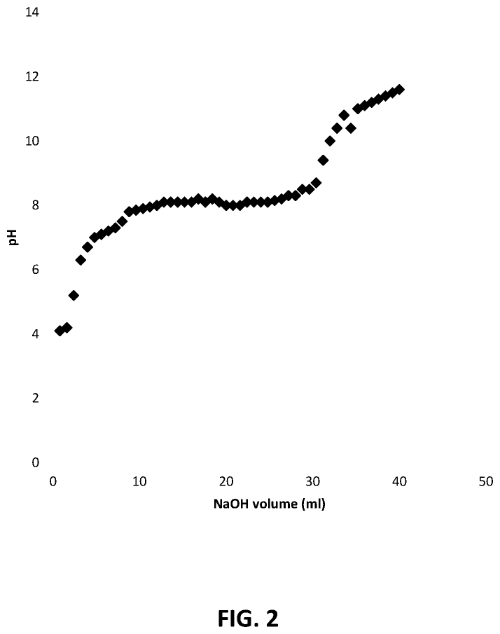

As shown in FIG. 2, the titration curve of the Eudragit L30D55 obtained from direct pH-titration revealed two equivalence which is in agreement with a polyacid character of the Eudragit L30D55 and it was observed irrespective of the polymer titration amount. The polymer may not be soluble until the pH of first equivalence since the solutions remained turbid.

When a first equivalence is reached, the polymer is not totally solubilized, which means that a dissociation carboxylic groups amount is not sufficient to ensure a total solubilization of the polymer and the medium remains turbid. During a second equivalence, the solution becomes translucent indicating that the amount of dissociated carboxylic functions is enough to ensure the solubilization of the polymer.

The second equivalence may be used to estimate an amount of carboxylic functions on the polymer. This amount varies with changing the source of methacrylic acid ethyl acrylate copolymer (Eudragit). For Eudragit L30D55, an estimated amount of carboxylic acid functional groups on the polymer was calculated as 6-mmol/g.

A total amount of alkaline reagent to be used for each formulation may be calculated based on the titration of water insoluble polymer (metacrylic acid ethyl acrylate copolymer).

### In Vitro Dissolution Studies

Using a United States Pharmacopeia (USP) apparatus 2 at 50 rpm, in 900 ml of medium at 37° C. with a wire sinker was used. Two hours of exposure in 0.1 N HCl (pH=1.2) was followed by testing in 0.05 M phosphate buffer of pH 6.8. The capsules that adhere to USP dissolution were accepted as enteric capsules. Capsules may not release more than 10% of the fill during two hours in 37° C. simulated gastric fluid. Capsules may be fully dissolved by 45 min in simulated intestinal fluid.

The foregoing is considered as illustrative only of the principles of the invention. Further, since numerous changes and modifications will readily occur to those skilled in the art, it is not desired to limit the invention to the exact construction and operation shown and described, and accordingly, all such suitable changes or modifications in structure or operation which may be resorted to are intended to fall within the scope of the claimed invention.

1. A soft gel capsule comprising:
30% wt. to 45% wt. water;
15% wt. to 19% wt. glycerol; and
a gel mass composition comprising a gelling agent and an alkaline agent.

2. The soft gel capsule according to claim 1, wherein the gelling agent is selected from at least one of: a gelatin and a vegetarian gelling agent.

3. The soft gel capsule according to claim 2, wherein the gelatin is 25% wt. to 35% wt.

4. The soft gel capsule according to 2, wherein the vegetarian gelling agent comprises at least one of: a tapioca, a pullulan, a hydroxypropyl methylcellulose (HPMC), a hydroxypropyl methylcellulose phthalate (HPMCP), a Eudragit L100, and a Eudragit L30D55.

5. The soft gel capsule according to claim 4, wherein the HPMCP is 15% wt. to 17.5% wt.

6. The soft gel capsule according to claim 4, wherein the HPMC is 25% wt. to 27% wt.

7. The soft gel capsule according to claim 4, wherein the Eudragit L100 is 15% wt. to 18% wt.

8. The soft gel capsule according to claim 6, wherein the Eudragit L100 is 15% wt.

9. The soft gel capsule according to claim 4, wherein the Eudragit L30D55 is 15% wt. to 46.5% wt.

10. The soft gel capsule according to claim 6, wherein the Eudragit L30D55 is 15% wt. to 46.5% wt.

11. The soft gel capsule according to claim 1, wherein the alkaline agent comprises 2.25 g to 3.95 g of NaOH.

12. The soft gel capsule according to claim 1, wherein the alkaline agent comprises 3.8 ml to 6.65 ml of a 25% solution of NH₄OH.

13. The soft gel capsule according to claim 1, further comprising: 1.8% wt. triethlycitrate.

14. A method of manufacturing a soft gel capsule comprises:
dissolving a gelling polymer into water to form an enteric polymer solution;
mixing an acid insoluble polymer with an alkali agent to form a film forming polymer; and
adding the film forming polymer to the enteric polymer solution while mixing and heating at 70° C. until a gel mass forms.

15. The method of manufacturing the soft gel capsule of claim 14 further comprises:
removing bubbles from the gel mass by maintaining the gel mass at 50° C. for about 24 hours.

16. The method of manufacturing the soft gel capsule of claim 14 further comprises:
removing bubbles from the gel mass by placing the gel mass under vacuum for about 18 hours.

17. The method of manufacturing the soft gel capsule of claim 14 further comprises:
titrating the acid insoluble polymer with concentrations of the alkali agent;
determining a first equivalence and a second equivalence from a titration curve, whereby the second equivalence corresponds to when the film forming polymer becomes translucent; and
selecting the second equivalence to determine an amount of the alkali agent.
